(19) United States
(12) Patent Application Publication
(10) Pub. No.: US 2011/0218326 A1
(43) Pub. Date: Sep. 8, 2011

(54) PLANT-PROTEIN PRODUCT AND METHOD FOR PREPARING THE SAME

(75) Inventors: Chuh-Hsiang Chen, Luzhu Township (TW); Chih-Yi Lu, Xinzhuang City (TW); Fa-Tzu Chen, Hsinchu City (TW)

(73) Assignee: ZEN-U BIOTECHNOLOGY CO., LTD., Taoyuan City (TW)

(21) Appl. No.: 12/716,376

(22) Filed: Mar. 3, 2010

(51) Int. Cl. C07K 14/435; C07K 1/14

(52) U.S. Cl. 530/370; 435/272

(57) ABSTRACT

A plant-protein product and the method for preparing the same are provided. The plant-protein product is obtained from a plant-protein raw material which is treated by a single-cell separation technique, and the method for preparing the plant-protein product comprises steps of providing the plant-protein raw material; and treating the plant-protein raw material by the single-cell separation technique to prepare the plant-protein product.

### FIELD OF THE INVENTION

The present invention relates to a plant-protein product and the method for preparing the same, and more particularly to a plant-protein product and the method for preparing the same by using the single-cell separation technique.

### BACKGROUND OF THE INVENTION

The method of preparing the fodder protein usually is a traditional fermentation or a dry press. The fermentation using microorganisms is a means of batch production whose time is very long (more than 8 hours) and quality is not stable. The crude protein content is 44˜50%, and the true digestibility is 90˜94%. Some manufacturers will mix animal-protein such as animal viscera which have high protein content to improve the crude protein content rate, but there are doubts of being polluted by disease and spoiled. Another common manufacturing method is the dry press, which is a physical means of continuous press production. The yield is 0.2 metric ton per hour; the quality of the product is more stable; and the crude protein content is 42˜48%. However, the true digestibility is only 85%, the processing cost is high, and the anti-nutritional factors can not be removed in the production process. In view of the drawbacks in the prior arts, the present invention provides a manufacturing method which can resolve the above-mentioned drawbacks.

### SUMMARY OF THE INVENTION

In accordance with one aspect of the present invention, a method for preparing a plant-protein product is provided. The method for preparing a plant-protein product comprises steps of providing a plant-protein raw material; and treating the plant-protein raw material by a single-cell separation technique to prepare the plant-protein product.

Preferably, the plant-protein raw material has a cell wall, and the single-cell separation technique is a process of cell wall removal by using an enzyme.

Preferably, the plant-protein raw material has a cell wall, and the single-cell separation technique is a protein micronization procedure being one selected from a group consisting of a high pressure homogenized procedure, a grinding procedure, a ball milling procedure and a high speed powder collision procedure to rupture the cell wall of the plant-protein raw material.

Preferably, a particle size of the plant-protein product after being treated by the single-cell separation technique is less than 100 μm.

Preferably, the method further comprises a step of processing the plant-protein product after being treated by the single-cell separation technique by a fix-site hydrolysis.

Preferably, the fix-site hydrolysis is performed by using an enzyme fermented from a microorganism.

Preferably, wherein the microorganism is one of Bacillus subtilis and Bacillus subtilis natto.

Preferably, the plant-protein raw material comprises one selected from a group consisting of a legume, an algae, a grain, a single-cell plant, other plant material and a combination thereof.

Preferably, the legume comprises one selected from a group consisting of a soybean, a black soybean, a sesame, a flax and a mung bean.

Preferably, the algae comprises one selected from a group consisting of a kelp, a layer, a wakame, a green algae and a spirulina.

Preferably, the grain comprises one selected from a group consisting of a rice, a millet, a maize, a barley, a wheat and an oats.

Preferably, the single-cell plant comprises a yeast cell.

Preferably, the other plant material comprises one selected from a group consisting of a soybean pomace, a colza meal, a cotton seed pomace and a glutenin.

In accordance with another aspect of the present invention, a plant-protein product is provided. A plant-protein product is obtained from a plant-protein raw material which is treated by a single-cell separation technique and a fix-site hydrolysis.

Preferably, the single-cell separation technique comprises a process of cell wall removal.

Preferably, the plant-protein raw material has a cell wall, and the single-cell separation technique comprises a protein micronization procedure being one selected from a group consisting of a high pressure homogenized procedure, a grinding procedure, a ball milling procedure and a high speed powder collision procedure to rupture the cell wall of the plant-protein raw material.

Preferably, the fix-site hydrolysis is performed by using an enzyme fermented from a microorganism, and the hydrolyzed plant-protein product has a peptide molecular weight being not larger than 10 KD.

Preferably, the microorganism is one of Bacillus subtilis and Bacillus subtilis natto.

In accordance with a further aspect of the present invention, a plant-protein product is provided. A plant-protein product is obtained from a plant-protein raw material which is treated by a single-cell separation technique.

Preferably, the plant-protein product is further treated by a fix-site hydrolysis by using an enzyme fermented from one of Bacillus subtilis and Bacillus subtilis natto.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows the diagram of preparing the plant-protein according to a preferred embodiment of the present invention;

FIG. 2 shows the diagram of preparing the plant-protein product according to a preferred embodiment of the present invention; and

FIG. 3 shows the diagram of the true digestibility under different treatments.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only it is not intended to be exhaustive or to be limited to the precise form disclosed.

The present invention relates to a method for preparing a plant-protein product. The plant-protein raw material includes a single-cell plant such as a yeast cell, a green algae or a spirulina, a multi-cell plant such as a soybean, a black soybean, a sesame, a flax, a mung bean, a rice, a millet, a maize, a barley, a wheat, an oats, a kelp, a laver or a wakame, a soybean pomace, a colza meal, a cotton seed pomace and a glutenin. All the plants have the cell wall, which would affect the digestibility of the nutrition. Thus, after the plant-protein raw material is treated by the single-cell separation technique to remove or rupture the cell wall thereof, the digestibility of the nutrition can be improved. The method for making the cell wall ruptured and separated can be any one of a high pressure homogenized procedure, a grinding procedure, a ball milling procedure, a high speed powder collision procedure and any other method which can rupture the cell wall of the plant-protein raw material. The cellulase can be used to remove the cell wall to make the particle size of the plant-protein product less than 100 μm. At this time, the digestibility of the plant-protein can be raised to 90˜95%.

Once the cell wall of the plant-protein raw material is ruptured, the digestive juice of the animal can direct hydrolyze the material inside the cell wall. In the present invention, the plant-protein is processed by a fix-site hydrolysis, and the hydrolyzed plant-protein product has a peptide molecular weight being not larger than 10 KD. At present, the digestibility of the plant-protein product can be raised to 98˜100%. The fix-site hydrolysis is a method of using specific enzyme to hydrolyze specific receptor protein. Through the specific hydrolysis, the protein is hydrolyzed to peptides having special structures. The peptide products have a specific function and flavor, and thus can be called functional peptides. The functional peptides not only replace the conventional animal fodder protein raw material, but can be applied in the development and application of the clinical replacement meal and functional foods for diabetes or metabolic diseases.

After the procedures of the single-cell separation technique and the fix-site hydrolysis, the anti-nutritional factors can be removed to enhance the digestibility after taken by the animal. The anti-nutritional factors include a trypsin inhibitor, a saponin, a phytic acid, a hemagglutinin, etc. which affect digestibility. In additional, due to using plant-protein as raw material source, the prepared plant-protein product doesn't have problems of tending to be spoiled like animal-protein, nor have any peculiar smell, and the freshness valuations are near 0. The freshness valuation standard in the present invention includes the flavor, acid value (AV), peroxide value (POV), TAB value and volatile basic nitrogen (VBN), wherein the acid value is a freshness index of fat, and the TAB value and VBN are freshness indexes of protein. Thus, the freshness valuation standard is that the lower the value is, the higher the freshness is.

The present invention provides a plant-protein product and the method for preparing the same. A process of cell wall removal being one of single-cell separation techniques comprises the steps of a plant-protein raw material being first under dry treatment; a water as a solvent and a cellulase being added therein; the pH value thereof being controlled between 8˜9; and shaking the system well to remove the cell wall of the plant-protein raw material. Besides, a protein micronization procedure being also one of single-cell separation techniques ruptures the cell wall of the plant-protein raw material by a physical means such as a high pressure homogenized procedure, a grinding procedure, a ball milling procedure and a high speed powder collision procedure. Both of the above-mentioned single-cell separation techniques can remove or rupture the cell wall of the plant-protein raw material to make the substances inside the cell exposed, and the specific enzyme can work therein to produce small peptide fragments.

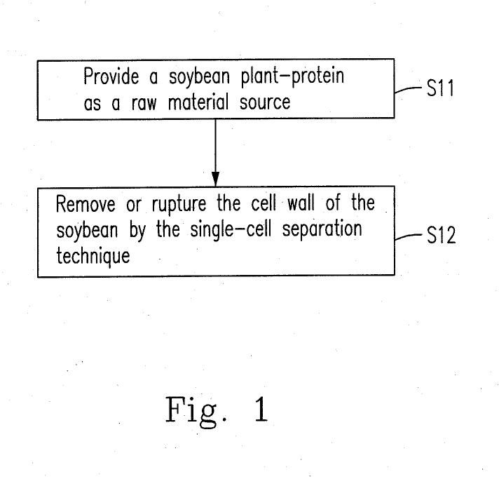

Please refer to FIG. 1, which shows the diagram of preparing the plant-protein according, to a preferred embodiment of the present invention. In the step S11, a soybean plant-protein is provided as a raw material source. In the step S12, the cell wall of the soybean is removed or ruptured by the single-cell separation technique, and the protein obtained therefrom is named Fprotein-1.

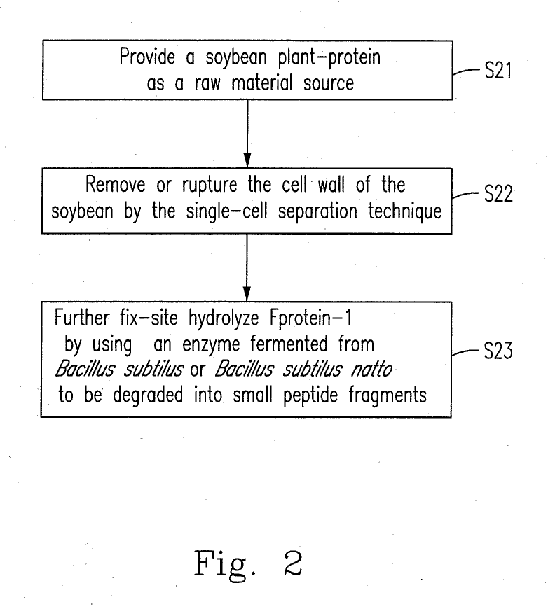

Please refer to FIG. 2, which shows the diagram of preparing the plant-protein product according to a preferred embodiment of the present invention. In the step S21, a soybean plant-protein is provided as a raw material source. In the step S22, the cell wall of the soybean is removed or ruptured by the single-cell separation technique, and the protein obtained therefrom is named Fprotein-1. In the step S23, Fprotein-1 is further fix-site hydrolyzed by using an enzyme fermented from Bacillus subtilis or Bacillus subtilis natto to be degraded into small peptide fragments, which is named Fprotein-2.

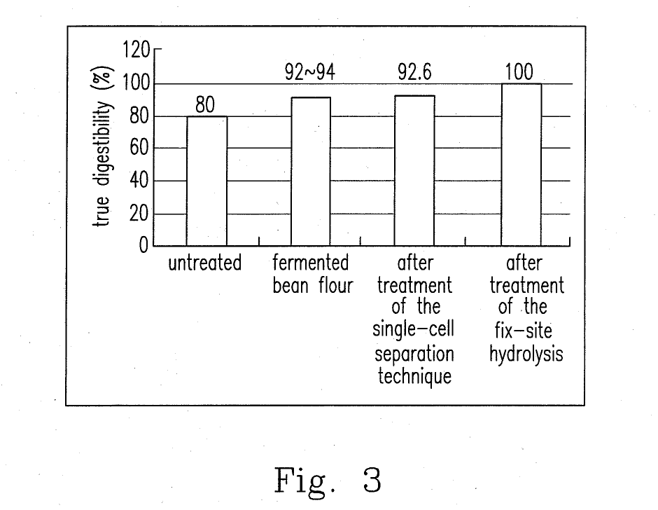

The true digestibility of the soybean itself is about 80%. After the cell wall of the soybean is removed or ruptured by the single-cell separation technique, the true digestibility of Fprotein-1 can raise 10˜15% from 80% to 90˜95%. If the specific enzyme fermented from Bacillus subtilis or Bacillus subtilis natto is additionally used to hydrolyze Fprotein-1, the true digestibility of Fprotein-2 will break through 90˜95% and reach 100%. FIG. 3 shows the diagram of the true digestibility under different treatments.

Besides, the crude protein content of Fprotein-2 reaches between the primary and the secondary fish meals of CNS. Since the true digestibility of Fprotein-2 has reached 100%, thus the digestible true protein thereof has surmounted that of the primary fish meal. Table 1 shows the comparison between the CNS standard of the fish meal and Fprotein-2, and table 2 shows the comparison between Fprotein-2 and major fodder proteins.

TABLE 1

| | Crude protein % | | Digestible true |
|---|---|---|---|
| | (CNS) | True digestibility % | protein % |
| Primary | >60 | Average 92.9 | 55.7 |
| Secondary | >50 | | 46.5 |
| Tertiary | >46 | | 42.3 |
| Fprotein-2 | 52~63 | 100 | 52~63 |

(digestible true protein % = crude protein*true digestibility)

TABLE 2

| | | True | |
|---|---|---|---|
| | Crude protein | digestibility | |
| | % | % | Anti-nutritional factor |
| Fprotein-2 | 52~63 | ~100 | None |
| Animal-protein (fish | 46~70 | 92~95 | histamine, cadaverine, |
| meal) | | | Salmonella |
| Soybean pomace | 42~48 | 80~85 | trypsin inhibitor, |
| powder untreated | | | agglutinin, saponin, |
| with the technique | | | mannanoligosaccharides, |
| | | | phytic acid |
| Fermented bean | 44~50 | 90~94 | low (unknown) |
| flour | | | |
| Distillers dried | 27 | 70 | gluten protein, crude |
| grains with soluble | | | fiber |

Besides nutritional contents (crude protein), freshness is the most important standard for the fodder protein classification. Bad freshness tends to make the livestock infected with disease and suffer from dyspepsia. Even if the crude protein content is high, bad freshness would lead to low utilization which makes the real utilizable nutrition (true protein) content low. Therefore, high quality fodder protein has not only good nutrition, but good freshness thereof is a necessary condition. The valuation index of freshness includes the flavor, acid value (AV), peroxide value (POV), TAB value and volatile basic nitrogen (VBN), wherein the acid value is a freshness index of fat, and the TAB value and VBN are freshness indexes of protein. The values of all the indexes are as low as well. In the present invention, the raw material sources are all plant-protein without any peculiar smell, which conform to the stipulation of freshness of the Japanese top grade fish meal, as shown in Table 3. There is no problem of tending to be spoiled like animal-protein, and each freshness valuation and the total value are all near 0, wherein the freshness valuation is the best, as shown in Table 4 which shows the comparison of freshness valuations between the hydrolyzed protein product and main commercial fish meals

TABLE 3

| | Flavor |
|---|---|
| Top grade | without oil odor, spoiled odor, mold odor and other odor |
| Primary | without oil odor, spoiled odor, mold odor and other odor, but a |
| | little scorched odor |
| Secondary | a little oil odor, corpse odor, spoiled odor, mold odor and other |
| | odor |
| Fprotein-2 | without oil odor, spoiled odor, mold odor and other odor, even |
| | without scorched odor |

(P.S.: the classification in CNS includes the primary, secondary and tertiary, but that in Japan includes the top grade, primary and secondary)

TABLE 4

| | | POV | TAB | VBN | Total |
|---|---|---|---|---|---|
| | AV (unit) | (unit) | (unit) | (mg/100 g) | value |
| Chile red fish meal | 29.3 | 9.40 | 13.10 | 97.60 | 167.30 |
| Peru red fish meal | 38.70 | 5.50 | 24.10 | 89.80 | 158.00 |
| Denmark red fish | 40.70 | 11.10 | 10.00 | 94.90 | 156.70 |
| meal | | | | | |
| Japan white fish | 26.30 | 25.90 | 7.50 | 88.10 | 137.20 |
| meal | | | | | |
| Fprotein-2 | ~0 | ~0 | ~0 | ~0 | ~0 |

Based on the above, the present invention effectively solves the problems and drawbacks in the prior art, and thus it fits the demand of the industry and is industrially valuable.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for preparing a plant-protein product, comprising steps of:
providing a plant-protein raw material; and
treating the plant-protein raw material by a single-cell separation technique to prepare the plant-protein product.

2. A method as claimed in claim 1, wherein the plant-protein raw material has a cell wall, and the single-cell separation technique is a process of cell wall removal by using an enzyme.

3. A method as claimed in claim 1, wherein the plant-protein raw material has a cell wall, and the single-cell separation technique is a protein micronization procedure being one selected from a group consisting of a high pressure homogenized procedure, a grinding procedure, a ball milling procedure and a high speed powder collision procedure to rupture the cell wall of the plant-protein raw material.

4. A method as claimed in claim 1, wherein a particle size of the plant-protein product after being treated by the single-cell separation technique is less than 100 μm.

5. A method as claimed in claim 4, further comprising a step of processing the plant-protein product after being treated by the single-cell separation technique by a fix-site hydrolysis.

6. A method as claimed in claim 5, wherein the fix-site hydrolysis is performed by using an enzyme fermented from a microorganism.

7. A method as claimed in claim 6, wherein the microorganism is one of Bacillus subtilis and Bacillus subtilis natto.

8. A method as claimed in claim 1, wherein the plant-protein raw material comprises one selected from a group consisting of a legume, an algae, a grain, a single-cell plant, other plant material and a combination thereof.

9. A method as claimed in claim 8, wherein the legume comprises one selected from a group consisting of a soybean, a black soybean, a sesame, a flax and a mung bean.

10. A method as claimed in claim 8, wherein the algae comprises one selected from a group consisting of a kelp, a layer, a wakame, a green algae and a spirulina.

11. A method as claimed in claim 8, wherein the grain comprises one selected from a group consisting of a rice, a millet, a maize, a barley, a wheat and an oats.

12. A method as claimed in claim 8, wherein the single-cell plant comprises a yeast cell.

13. A method as claimed in claim 8, wherein the other plant material comprises one selected from a group consisting of a soybean pomace, a colza meal, a cotton seed pomace and a glutenin.

14. A plant-protein product, obtained from a plant-protein raw material which is treated by a single-cell separation technique and a fix-site hydrolysis.

15. A product as claimed in claim 14, wherein the single-cell separation technique comprises a process of cell wall removal.

16. A product as claimed in claim 14, wherein the plant-protein raw material has a cell wall, and the single-cell separation technique comprises a protein micronization procedure being one selected from a group consisting of a high pressure homogenized procedure, a grinding procedure, a ball milling procedure and a high speed powder collision procedure to rupture the cell wall of the plant-protein raw material.

17. A product as claimed in claim 14, wherein the fix-site hydrolysis is performed by using an enzyme fermented from a microorganism, and the hydrolyzed plant-protein product has a peptide molecular weight being not larger than 10 KD.

18. A product as claimed in claim 17, wherein the microorganism is one of Bacillus subtilis and Bacillus subtilis natto.

19. A plant-protein product, obtained from a plant-protein raw material which is treated by a single-cell separation technique.

20. A product as claimed in claim 19, being further treated by a fix-site hydrolysis by using an enzyme fermented from one of Bacillus subtilis and Bacillus subtilis natto.
